(19) United States
(12) Patent Application Publication
(10) Pub. No.: US 2011/0190856 A1
(43) Pub. Date: Aug. 4, 2011

(54) Garment and Method for Treating Fatty Deposits on a Human Body

(75) Inventors: Jamie T. Burke, Vienna, VA (US); Lark T. MacPhail, Owings Mill, MD (US)

(73) Assignee: FreezeAwayFat LLC, Vienna, VA (US)

(21) Appl. No.: 13/021,358

(22) Filed: Feb. 4, 2011

Related U.S. Application Data
(60) Provisional application No. 61/301,347, filed on Feb. 4, 2010.

(51) Int. Cl. A61F 7/10; A41D 1/06; F25D 31/00; F25C 1/00

(52) U.S. Cl. 607/112; 2/228; 62/259.3; 62/66

(57) ABSTRACT

A garment for treating white and brown fat areas on a human body employs a wearable garment with pockets for holding cooling inserts in positions to cool fat of a wearer. The pockets are configured at predetermined locations on a wearable garment adjacent to areas of the body having white adipose tissue (fatty deposits) to be treated by cooling. The pockets receive cooling inserts that stimulate brown adipose tissue to consume the fatty deposits. Preferably, the wearable garment is a high-waisted pair of shorts made with elastic material. The pockets are preferably located on the thighs, buttocks, stomach, hips, waist and lower back of a wearer, where fat is often a problem. A method involves positioning cooling means on a garment at one or more locations corresponding to one or more fatty deposits to be treated, positioning the cooling means adjacent the fatty deposits based upon the user wearing the garment, and cooling the fatty deposits with the cooling means to a temperature sufficient to stimulate brown fat to burn calories and compromise white fat cells by crystallization of cytoplasmic lipid deposits within the white fat cells.

### RELATED APPLICATIONS

This application claims the benefit of U.S. Provisional Application Ser. No. 61/301,347 filed Feb. 4, 2010, which is hereby incorporated by reference in its entirety for all purposes and specifically for the concept and structure of a garment for different parts of the body having pockets strategically located for fat reduction in specific areas of the body. The structure and functions of the pockets in the garment and their method of affixing to the garment (permanent and removable) is incorporated herein. The concept of form fitting cold packs that may comprise a gel or gel beads or other conformable cold-application devices is also incorporated herein.

### BACKGROUND

Recent research has demonstrated that brown fat cells and white fat cells are uniquely sensitive to cold temperatures. Researchers claim in the New England Journal of Medicine that adults may retain brown adipose tissue (sometimes referred to herein as “brown fat”). The primary purpose of brown fat is to generate heat when the body experiences cold temperatures. When activated by cold temperatures, brown fat can expend tremendous amounts of energy, which burn a huge numbers of calories. This results in the shedding of fat.

Subcutaneous white fat, located beneath the skin, is often visible on the exterior of the body, and can appear as lumpy deposits or “blobs.” White fat cells are made up primarily of lipids. Recent studies have demonstrated that localized cooling subcutaneous white fat results in crystallization of cytoplasmic lipid deposits within white fat cells. The crystallization of lipids has been shown to induce cell death (sometimes referred to herein as “apoptosis”). Once damaged by cold temperatures following apoptosis, immune cells envelope the compromised white fats cells and eventually flush them out of the body through the natural process of elimination. Thus, cold temperatures not only help to activate brown fat, but studies show it can reduce the volume of white fat.

People all over the world continue to spend many hours and significant funds on ridding themselves of fat congregating on their thighs, hips, waist, buttocks and stomach area. Numerous fat reduction treatments have been available for years, including but not limited to creams, lotions, massages, radio frequency treatment, liposuction, etc., most in attempts to improve circulation and flush out the stubborn fat. Treatments to reduce stubborn fatty deposits on the body which are resistant to diet and exercise are part of a global beauty industry with annual retail sales approaching $250 Billion (as reported by the Personal Care Association).

Those plagued by stubborn fatty deposits can sometimes be shy about wearing bathing suits and shorts or even snugly fitted knitwear. Many are determined to get rid of it and will diet, exercise, apply lotions, get massages, and even resort to surgery to have slimmer thighs, hips and stomach.

Taking diet pills and resorting to surgery exposes an individual to a variety of risks, including adverse reactions to medications and impurities found in over the counter remedies, as well as infections and scarring.

### SUMMARY

Embodiments are directed to garments that apply cold to regions of the body to both stimulate brown fat to consume calories and to compromise white fats cells resulting in a loss of fat in those regions.

### DESCRIPTION OF THE DRAWINGS

FIG. 1 is a block diagram illustrating a front view of a garment according to an embodiment.

FIG. 2 is a block diagram illustrating a back view of a garment according to an embodiment.

FIG. 3 is a block diagram illustrating a cooling insert according to an embodiment.

FIG. 4 is a diagram illustrating a front view of a garment according to an embodiment.

FIG. 5 is a diagram illustrating a back view of a garment according to an embodiment.

FIG. 6 is a diagram illustrating a front view of a garment according to another embodiment.

FIG. 7 is a diagram illustrating a back view of a garment according to another embodiment.

FIG. 8 illustrates an embodiment being worn by a user.

FIG. 9 is a diagram illustrating a pair of cooling inserts according to an embodiment.

FIG. 10 is a flow chart of an embodiment.

### DETAILED DESCRIPTION

Embodiments are directed to garments that apply cold to fatty regions of the body to both stimulate brown fat to consume calories and to compromise white fats cells resulting in a loss of fat in those regions.

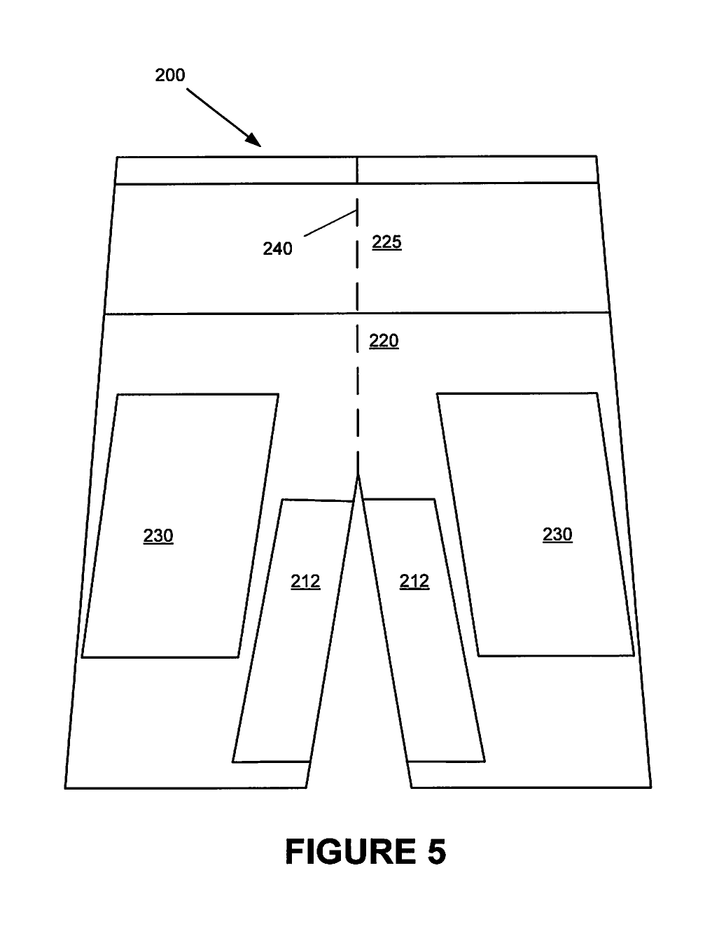

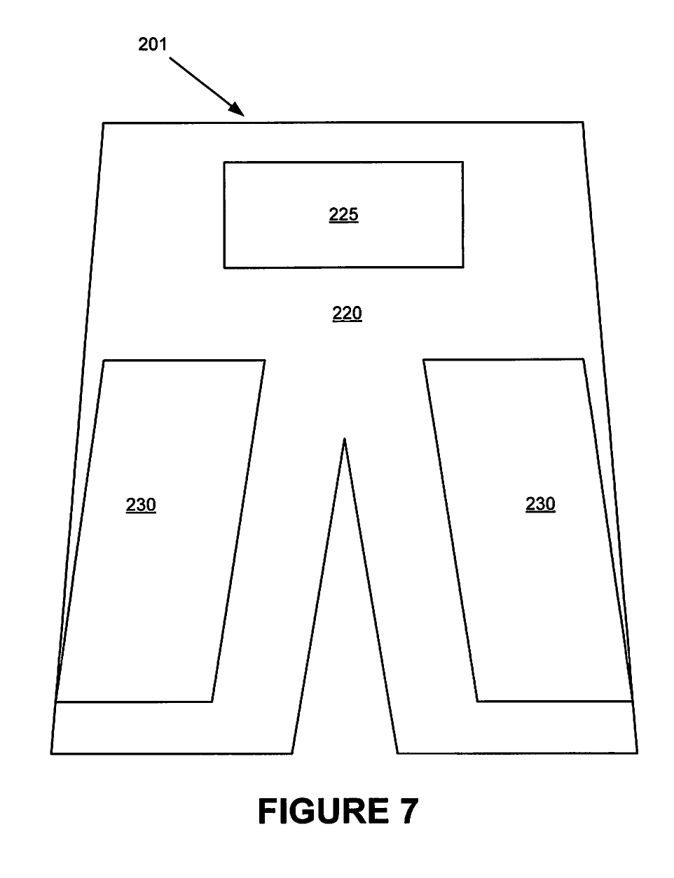

As used herein, the terms “thigh,” “inner thigh,” and “outer thigh” have their ordinary meaning, where inner and outer refer to positions generally relative to a centerline on the front of a thigh, but “outer thigh” can also include frontal portions of a thigh. The term “buttocks” refers to areas including the buttocks in accordance with its ordinary meaning, as well as areas adjacent thereto, including the hip and rear of the thigh, as illustrated generally by reference numerals 230 of FIGS. 5 and 7. The terms “waist,” “stomach” and “lower back” refer to areas including the waist, stomach and lower back, respectively, in accordance with their ordinary meanings, as well as areas adjacent thereto, including the side areas (sometimes referred to as “love handles”) at the sides of a wearer.

The drawings are merely to illustrate various embodiments and are not to scale. As such, the drawings are not to be interpreted as limiting the embodiments to any particular dimensions.

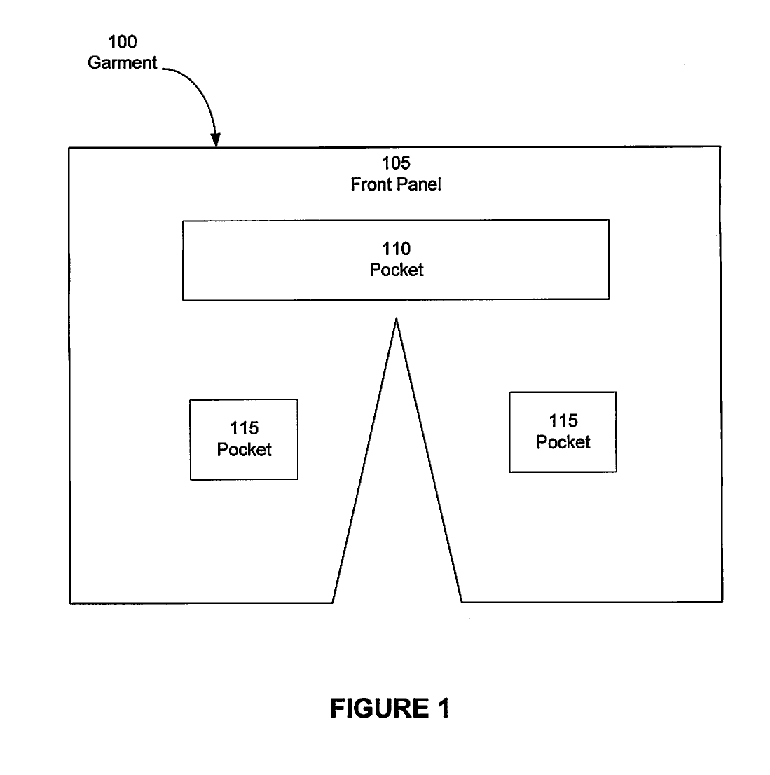

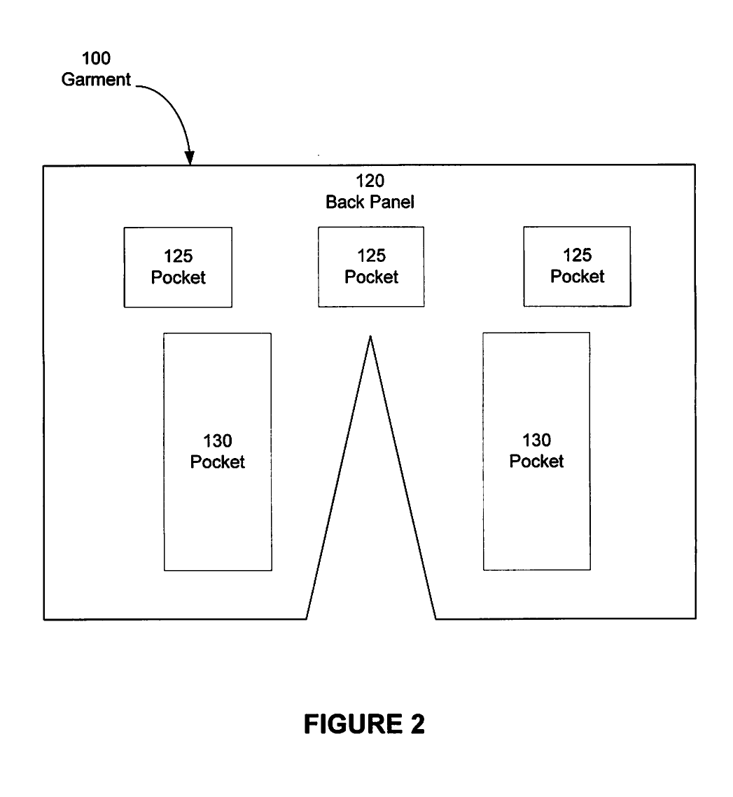

FIG. 1 illustrates an embodiment in which a cooling garment 100 is constructed as a pant. The front panel 105 is illustrated having pockets 110 and 115. FIG. 2 illustrates a back panel 120 having pockets 125 and 130. The size, shape, number and location of these pockets may vary based on the location of the fat to be treated and the size of that area.

In an embodiment, the pockets are not permanently integrated into the garment 100. Rather, the pockets may be affixed to the garment by fastening means such as fabric hook-and-loop fasteners, adhesives, or snaps. In this embodiment, the garment 100 may include pre-applied fastening means that allow pockets with complimentary fastening means to be attached to the garment 100 in a configuration determined by the wearer of the garment 100.

While the garment 100 is illustrated as a pant, other garment shapes are also possible. By way of illustration and not by way of limitation, a garment may be shaped as sleeves, a wrap, a vest, and a shirt. These other-shaped garments would also be provided with pockets as previously described. An other-shaped garment may be provided with attaching means to facilitate securing the other-shaped garment to the body in proximity to the fatty area to be treated. By way of illustration and not by way of limitation, the attaching means of an other-shaped garment may include fabric hook-and-loop fasteners, adhesives, snaps, belts, straps, and elastic bands. Pockets may be secured to the other-shaped garments as previously described.

In an embodiment, the garment 100 and garments of other shapes may be made of compression/elastic blend fabric blend. The elasticity of the garment may be adjusted to minimize the number of sizes required to fit the largest possible number of wearers.

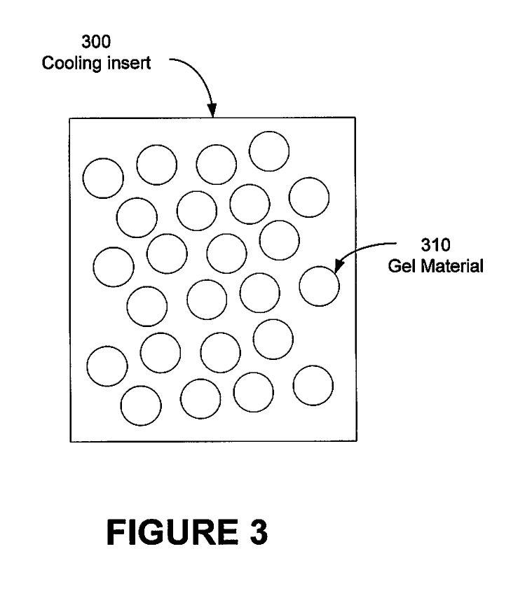

The pockets are configured to accommodate one or more cooling inserts as illustrated in FIG. 3. In an embodiment, a cooling insert 300 comprises a quantity of gel beads or any other form fitting moldable gel packs 305 that retain cold when cooled. The gel packs 305 may be suspended in a liquid that remains fluid at temperatures below 32 degrees Fahrenheit.

In use, the garment 100 is worn as pants and cooling packets 300 inserted into pockets adjacent to fatty deposits of the wearer. An other-shaped garment is secured to the wearer by the described attaching means to locate the pocket or pockets in proximity to the fatty area of the body to be treated.

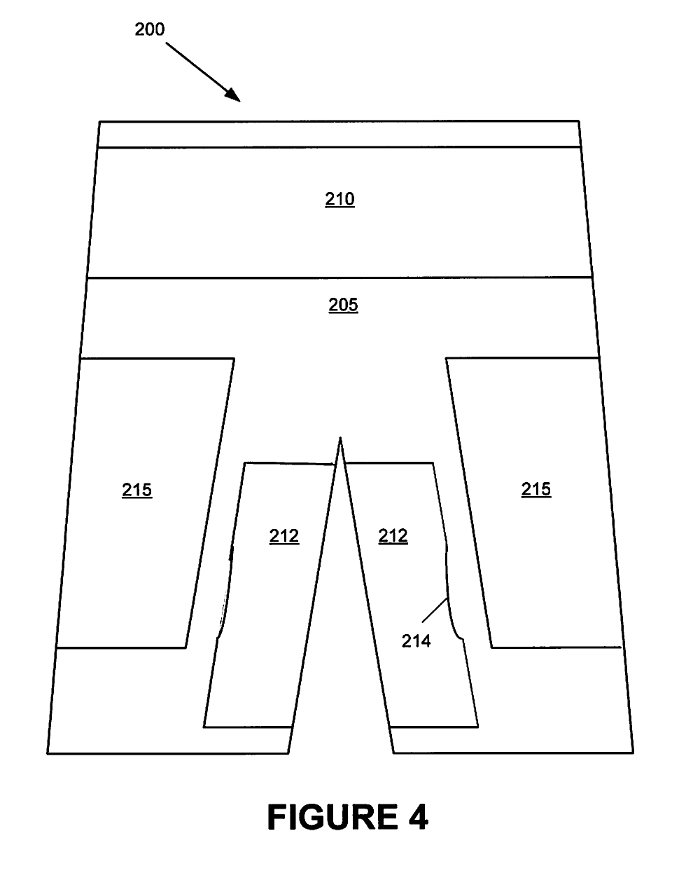

In another embodiment, a garment 200 is illustrated in FIGS. 4 and 5, and comprises a high-waisted pair of shorts (sometimes referred to as a “slimmer”). In this embodiment, the pockets for cooling inserts 300 are sewn or otherwise affixed to the outside of garment 200 in predetermined locations where fat will often be located so that a user that desires to treat fat in such locations can provide cooling thereto. Although placement of the pockets on the outside allows insertion and removal of cooling inserts 300 while the garment 200 is being worn by a user, it is also contemplated that the pockets can be placed on an interior of the garment 200.

FIG. 4 illustrates a front panel 205 of garment 200. The base garment 200 and the pockets are formed of an elastic material so as to conform to a user (i.e., wearer) and conform the cold inserts to maintain contact with the user. Such an elastic material further conforms to users of various sizes and allows the continued use of the garment by an individual even after the individual slims down (e.g., due to the present treatment or diet) or gains weight. Suitable elastic materials include fabrics of 82% Nylon/18% Spandex, 90% Polyester/10% Lycra and 83% Polyester/17% Lycra, although these examples are not meant as limitations. One of ordinary skill in the art will understand that various ranges of elastic fabrics and other fabric-like materials can provide the desired properties, with these materials referred to herein as elastic materials. Cotton gussets can be provided for stabilizing the garment, and overlock and flatlock seams can be used, as appropriate.

As illustrated in FIG. 4, leg portions of garment 200 include inner thigh pockets 212 located on a portion of the garment 200 that will contact an inner thigh of a user wearing the garment 200. Pockets 212 include vertical access openings 214 on a portion of a front edge to allow insertion and removal cold inserts 300. In one possible configuration, the inner thigh pockets 212 are dimensioned for use with cold inserts having dimensions of approximately 6″×9″×¼″ (˜15 cm×23 cm×0.64 cm).

The leg portions of garment 200 further include outer thigh pockets 215 adjacent to the inner thigh pockets 212 on a portion of the garment 200 that will contact an outer or front thigh portion of a user wearing the garment 200. While these outer thigh pockets 215 could also employ vertical access openings, in a preferred embodiment, the pockets 215 have open, elasticized tops that allow insertion and removal cold inserts 300. In one possible configuration, the inner thigh pockets 215 are dimensioned for use with cold inserts having dimensions of approximately 6″×9″×¼″ (˜15 cm×23 cm×0.64 cm). In general, fat on this portion of a thigh is located higher relative to the inner thigh, so the position of this outer thigh pocket 215 can be vertically higher relative to inner thigh pocket 212.

A trunk portion of garment 200 includes at least one stomach pocket 210 located on a portion of the garment 200 that will contact the stomach and/or waist area of a user when wearing the garment 200. The flexible nature of a garment made with an elastic material allows the location of the stomach pocket 210 to be adjusted up or down to match a location of fat locations on a user. In one possible configuration, the stomach pocket 210 has an open, elasticized top and extends across the entire front panel 205. Such a pocket 210 can be dimensioned for use with one or more cold inserts having dimensions of approximately 6″ (˜15 cm) in a vertical dimension.

FIG. 5 illustrates a back panel 220 of the garment 200 illustrated in FIG. 4. A trunk portion of garment 200 includes at least one lower back pocket 225 located on a portion of the garment 200 that will contact the lower back and/or waist area of a user when wearing the garment 200. The flexible nature of a garment made with an elastic material allows the location of the lower back pocket 225 to be adjusted up or down to match a location of fat locations on a user. In one possible configuration, the lower back pocket 225 has an open, elasticized top and extends across the entire back panel 220. Such a pocket 225 can be dimensioned for use with one or more cold inserts having dimensions of approximately 6″ (˜15 cm) in a vertical dimension. In one embodiment, the lower back pocket 225 and the stomach pocket 210 are formed as a single, circumferential pocket that begins at seam 240 on the back panel 220, extends in one direction across the back panel 220 to the front panel 205 (where the pocket becomes stomach pocket 210), across the front panel 205, and back to seam 240 from the other direction on back panel 220.

The inner thigh pockets 212 extend to the leg portions of back panel 220, as illustrated in FIG. 5. The leg portions of garment 200 further include buttocks pockets 230 adjacent to the inner thigh pockets 212 on a portion of the garment 200 that will contact the buttocks and rear thigh portion of a user wearing the garment 200. While these buttocks pockets 230 could also employ vertical access openings, in a preferred embodiment, the pockets 230 have open, elasticized tops that allow insertion and removal cold inserts 300. In one possible configuration, the buttocks pockets 230 are located immediately adjacent the outer thigh pockets 215 and are dimensioned for use with cold inserts having dimensions of approximately 6″×9″×¼″ (˜15 cm×23 cm×0.64 cm). In general, fat on this portion of a buttocks and thigh is located higher relative to the inner thigh, so the position of this buttocks pocket 230 can be vertically higher relative to inner thigh pocket 212.

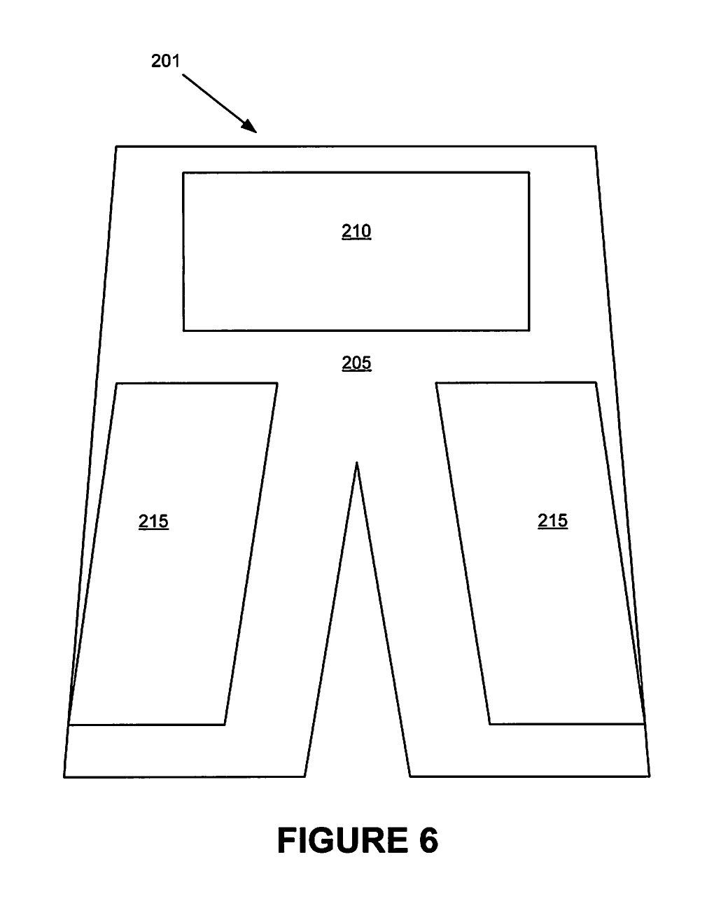

In another embodiment, a garment 201 is illustrated in FIGS. 6 and 7, and comprises a high-waisted pair of shorts (sometimes referred to as a “slimmer”). In this embodiment, the pockets for cooling inserts 300 are sewn or otherwise affixed to the garment 201 in predetermined locations where fat will often be located so that a user that desires to treat fat in such locations can provide cooling thereto. FIG. 6 illustrates a front panel 205 of garment 201. The base garment 201 and the pockets are formed of an elastic material so as to conform to a user (i.e., wearer) and conform the cold inserts to maintain contact with the user. Such an elastic material further conforms to users of various sizes and allows the continued use of the garment by an individual even after the individual slims down (e.g., due to the present treatment or diet) or gains weight. Elastic materials include fabrics of 82% Nylon/18% Spandex, 90% Polyester/10% Lycra and 83% Polyester/17% Lycra, although these examples are not meant as limitations. One of ordinary skill in the art will understand that various ranges of elastic fabrics and other fabric-like materials can provide the desired properties, with these materials referred to herein as elastic materials. Cotton gussets can be provided for stabilizing the garment, and overlock and flatlock seams can be used, as appropriate.

As illustrated in FIG. 6, leg portions of garment 200 include thigh pockets 215 located on a portion of the garment 200 that will contact a thigh of a user wearing the garment 201. Pockets 215 include access openings along an elasticized top edge for insertion and removal cold inserts 300. In one possible configuration, the thigh pockets 215 are dimensioned for use with cold inserts having dimensions of approximately 6″×9″×¼″ (˜15 cm×23 cm×0.64 cm).

A trunk portion of garment 201 includes at least one stomach pocket 210 located on a portion of the garment 201 that will contact the stomach area of a user when wearing the garment 201. The flexible nature of a garment made with an elastic material allows the location of the stomach pocket 210 to be adjusted up or down to match a location of fat locations on a user. In one possible configuration, the stomach pocket 210 has an open, elasticized top and is dimensioned for use with cold inserts having dimensions of approximately 4″×10″×¼″ (˜10 cm×25 cm×0.64 cm).

FIG. 7 illustrates a back panel 220 of the garment 201 illustrated in FIG. 6. A trunk portion of garment 201 includes at least one lower back pocket 225 located on a portion of the garment 201 that will contact the lower back area of a user when wearing the garment 201. The flexible nature of a garment made with an elastic material allows the location of the lower back pocket 225 to be adjusted up or down to match a location of fat locations on a user. In one possible configuration, the lower back pocket 225 has an open, elasticized top and is dimensioned for use with cold inserts having dimensions of approximately 3″×5″×¼″ (˜8 cm×13 cm×0.64 cm).

The leg portions of garment 201 further include buttocks pockets 230 on a portion of the garment 201 that will contact the buttocks and rear thigh portion of a user wearing the garment 201. While these buttocks pockets 230 could also employ vertical access openings, in a preferred embodiment, the pockets 230 have open, elasticized tops that allow insertion and removal cold inserts 300. In one possible configuration, the buttocks pockets 230 are dimensioned for use with cold inserts having dimensions of approximately 6″×9″×¼″ (˜15 cm×23 cm×0.64 cm).

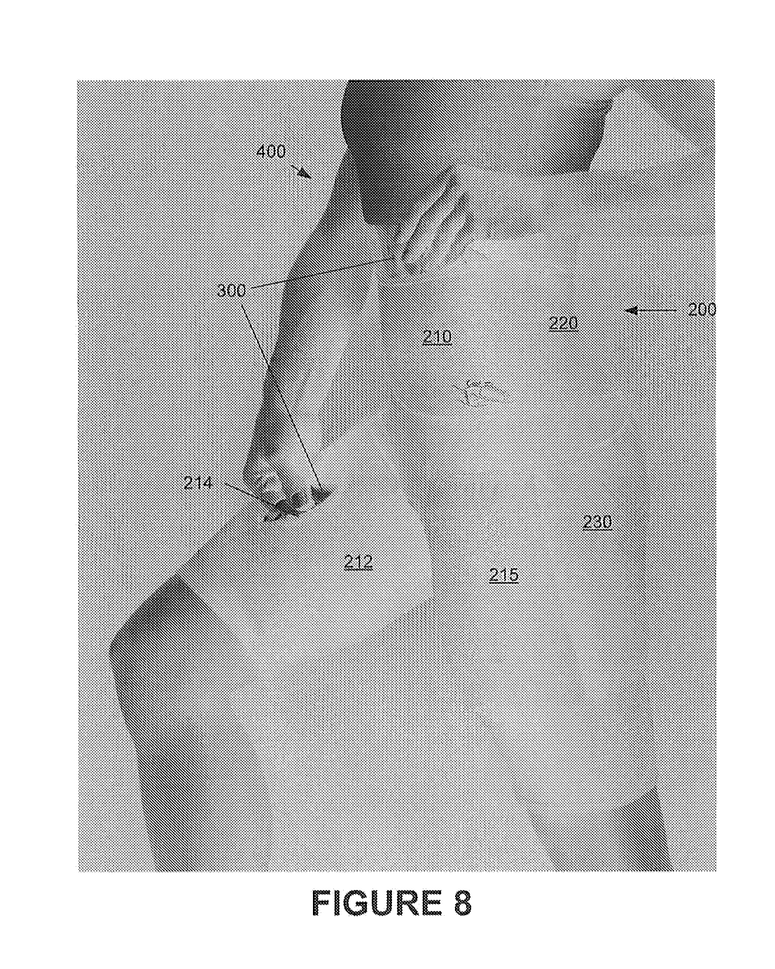

FIG. 8 illustrates an embodiment of a garment 200 generally corresponding to the embodiment of FIGS. 4 and 5 as worn by a user 400. FIG. 8 discloses a garment 200 in which cold inserts 300 are placed in inner thigh pocket 212, outer thigh pocket 215, buttocks pocket 230, and stomach pocket 210 (formed as a circumferential pocket with back pocket 220). Such a garment 200 is marketed by the present inventor under the tradename of Cool Shapes™ from FreezeAwayFat LLC of Baltimore, Md.

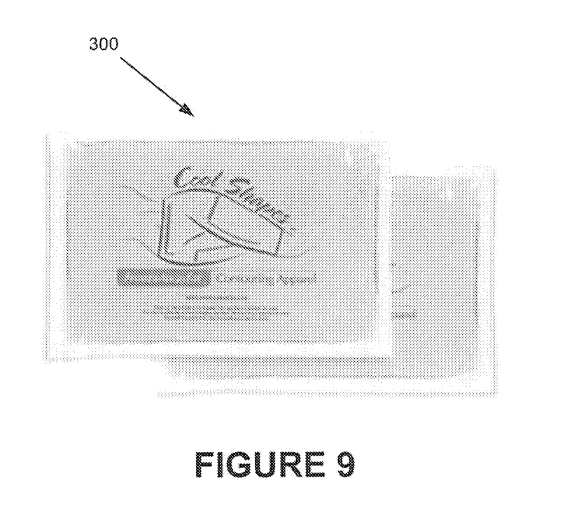

FIG. 9 illustrates a pair of cold inserts 300. In one embodiment, the cold inserts 300 are substantially rectangular and approximately ¼″ thick. The cold inserts can comprise a super absorbent polymer and water suspended in propylene glycol. The absorbed water can be frozen (at ˜32° F./0° C.) to form a phase-change material that absorbs heat in order to provide cooling (as typically found in cold packs used for injuries, to prevent swelling, and to ease pain). The propylene glycol allows the cold inserts 300 to remain flexible when frozen. The outside of cold inserts 300 is preferably a high-strength, puncture-resistant plastic. In use, the cold inserts 300 can be frozen by placement in a freezer at a temperature of approximately 30-34° F. (˜−1 to 1° C.). Suitable cold packs for use as the cold inserts 300 are available from Source One International of British Columbia, Canada, under the tradename of GelPax™ Embodiments are not limited to such cold inserts and can use any suitably thin and flexible material for application of cold temperatures in the desired temperature range. For example, small compartments of cooling or phase change material (PCM) positioned adjacent one another on a flexible substrate can also be used.

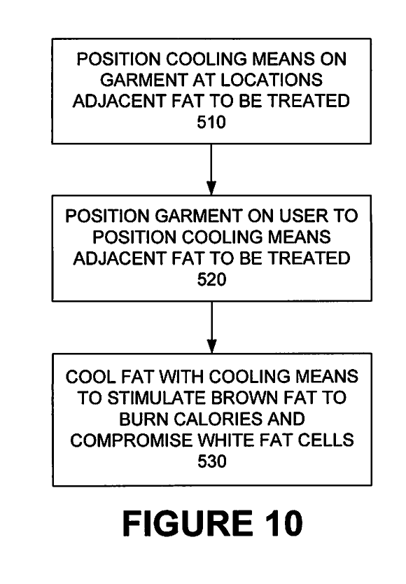

FIG. 10 illustrates a flow chart for a process embodiment for treating fatty deposits with cooling. In the process, cooling means are positioned on a garment at one or more locations corresponding to one or more fatty deposits to be treated, at 510. Cooling means can be a flexible thermal storage material, as discussed above with respect to the cooling inserts 300, or any other suitable cooling means that can be attached to a garment and will provide cool temperatures near the freezing point of water (0° C.). Other suitable cooling means include, but are not limited to, mixtures of alcohol and water, cold beads of various organic and inorganic materials, and substances that can be pre-cooled and which transfer heat (cold) gradually over an extended period of time. The garment is then worn by a user to position the cooling means adjacent the fatty deposits on the user to be treated, at 520. The fatty deposits are then cooled via the cooling means to a temperature sufficient to stimulate brown fat to burn calories and compromise white fat cells by crystallization of cytoplasmic lipid deposits within the white fat cells, at 530.

In one embodiment, the garment for applying cooling to fatty deposits of a wearer employs 6 pockets and consists essentially of a high-waisted pair of shorts formed from an elastic material with the pockets for 6 cooling inserts. The shorts are configured for encompassing the thighs, buttocks, hips, stomach, waist and lower back of the wearer and have a plurality of pockets formed at predetermined locations on the shorts. The 6 pockets consist of a thigh pocket formed of elastic material on each thigh, a buttock pocket formed of elastic material on each buttocks, a stomach pocket formed of elastic material on the stomach, and a lower back pocket formed of elastic material on the lower back. The cooling inserts are configured for insertion into at least one of each corresponding type of the plurality of pockets, and each cooling insert comprises a flexible thermal storage material, such as freezable gel.

Variations on this embodiment include those: wherein each of the plurality of pockets has a top-loading opening for insertion and removal of corresponding cooling inserts, wherein each thigh pocket, each buttocks pocket, and each corresponding cooling insert is approximately 6 inches (˜15 cm) wide and approximately 9 inches (˜23 cm) tall, wherein the stomach pocket and the corresponding cooling insert are each approximately 10 inches (˜25 cm) wide and approximately 4 inches (˜10 cm) tall, and wherein the lower back pocket and the corresponding cooling insert are each approximately 5 inches (˜13 cm) wide and approximately 3 inches (˜8 cm) tall.

Preferrably, each thigh pocket and each buttocks pocket are immediately adjacent one another, and/or each thigh pocket and each buttocks pocket are the same size and located immediately adjacent one another.

The flexible thermal storage material of each cooling insert in this embodiment preferably comprises beads of phase change material that freeze at approximately 0° C. (32° F.). In one variation of this embodiment, a corresponding cooling insert is positioned in each of the plurality of pockets.

In another embodiment, a garment for applying cooling to fatty deposits of a wearer employs 8 pockets and consists essentially of a high-waisted pair of shorts formed from an elastic material with pockets for 8 cooling inserts. The shorts are configured for encompassing the inner and outer thighs, buttocks, hips, stomach, waist and lower back of the wearer and have a plurality of pockets formed at predetermined locations on the shorts. The 8 pockets consist of an inner thigh pocket formed of elastic material on each inner thigh, an outer thigh pocket formed of elastic material on each outer thigh, a buttock pocket formed of elastic material on each buttocks, a stomach pocket formed of elastic material on the stomach, and a lower back pocket formed of elastic material on the lower back. The cooling inserts are configured for insertion into at least some of the 8 pockets, and each cooling insert comprises a flexible thermal storage material, such as freezable gel.

Variations on this embodiment include those: wherein each of the outer thigh pockets, buttocks pockets, stomach pocket and lower back pocket has a top-loading opening for insertion and removal of cooling inserts, wherein each of the inner thigh pockets has a side opening with a height substantially less than a height of the inner thigh pocket, wherein each inner thigh pocket, each outer thigh pocket, each buttocks pocket, and each corresponding cooling insert is approximately 6 inches (˜15 cm) wide and approximately 9 inches (˜23 cm) tall, wherein the stomach pocket and lower back pocket are configured to accommodate two cooling inserts approximately 6 inches (˜15 cm) tall and approximately 9 inches (˜23 cm) wide in a substantially side-by-side arrangement, and wherein the plurality of cooling inserts are approximately 6 inches (˜15 cm) by approximately 9 inches (˜23 cm).

In one preferred variation of this embodiment, each inner thigh pocket and each outer thigh pocket are immediately adjacent one another along a substantial portion of their height, and each outer thigh pocket and each buttocks pocket are immediately adjacent one another. In another preferred variation, each outer thigh pocket and each buttocks pocket are the same size and located immediately adjacent one another. In a further preferred embodiment, the stomach pocket and the lower back pocket are adjacent each other to extend substantially circumferentially around the waist of the wearer.

The flexible thermal storage material of each cooling insert in this embodiment preferably comprises beads of phase change material that freeze at approximately 0° C. (32° F.). In one variation of this embodiment, a corresponding cooling insert is positioned in each of the plurality of pockets.

A method for using such a garment to apply cooling to fatty deposits of a wearer so as to reduce the fatty deposits, comprises freezing the cooling inserts in a flat position for at least two hours, inserting the cooling inserts into the plurality of pockets adjacent to areas of the wearer having fatty deposits to be treated by cooling, and wearing the garment for at least ½ hour.

Another embodiment comprises a method of treating fatty deposits of a user with cooling that involves positioning cooling means providing a first temperature on a garment at one or more locations corresponding to one or more fatty deposits to be treated, positioning the cooling means adjacent the fatty deposits based upon the user wearing the garment, and cooling the fatty deposits with the cooling means to a second temperature sufficient to stimulate brown fat to burn calories and compromise white fat cells by crystallization of cytoplasmic lipid deposits within the white fat cells. The temperature involved in this method is preferably approximately 0° C., and the cooling means is preferably a flexible thermal storage material.

A garment for applying cooling to fatty deposits of a wearer and method of use to reduce fatty deposits has been described. It will be understood by those skilled in the art that the present invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. Further, any reference to claim elements in the singular, for example, using the articles “a,” “an,” or “the” is not to be construed as limiting the element to the singular.

What is claimed is:

1. A garment for applying cooling to fatty deposits of a wearer, consisting essentially of:
a high-waisted pair of shorts formed from an elastic material, the shorts configured for encompassing the thighs, buttocks, hips, stomach, waist and lower back of the wearer;
a plurality of pockets formed at predetermined locations on the shorts, the plurality of pockets consisting of:
a thigh pocket formed of elastic material on each thigh;
a buttock pocket formed of elastic material on each buttocks;
a stomach pocket formed of elastic material on the stomach; and
a lower back pocket formed of elastic material on the lower back; and
a plurality of cooling inserts configured for insertion into at least one of each corresponding type of the plurality of pockets, wherein each cooling insert comprises a flexible thermal storage material.

2. The garment of claim 1, wherein each of the plurality of pockets has a top-loading opening for insertion and removal of corresponding cooling inserts.

3. The garment of claim 1, wherein each thigh pocket, each buttocks pocket, and each corresponding cooling insert is approximately 6 inches (˜15 cm) wide and approximately 9 inches (˜23 cm) tall.

4. The garment of claim 1, wherein the stomach pocket and the corresponding cooling insert are each approximately 10 inches (˜25 cm) wide and approximately 4 inches (˜10 cm) tall.

5. The garment of claim 1, wherein the lower back pocket and the corresponding cooling insert are each approximately 5 inches (˜13 cm) wide and approximately 3 inches (˜8 cm) tall.

6. The garment of claim 3, wherein each thigh pocket and each buttocks pocket are immediately adjacent one another.

7. The garment of claim 1, wherein each thigh pocket and each buttocks pocket are the same size and located immediately adjacent one another.

8. The garment of claim 1, wherein the flexible thermal storage material of each cooling insert comprises phase change material that freezes at approximately 0° C. (32° F.) suspended in ethylene glycol.

9. The garment of claim 1, wherein a corresponding cooling insert is positioned in each of the plurality of pockets.

10. A garment for applying cooling to fatty deposits of a wearer, consisting essentially of:
a high-waisted pair of shorts formed from an elastic material, the shorts configured for encompassing the thighs, buttocks, hips, stomach, waist and lower back of the wearer;
a plurality of pockets formed at predetermined locations on the shorts, the plurality of pockets consisting of:
an inner thigh pocket formed of elastic material on each inner thigh;
an outer thigh pocket formed of elastic material on each outer thigh;
a buttock pocket formed of elastic material on each buttocks;
a stomach pocket formed of elastic material on the stomach; and
a lower back pocket formed of elastic material on the lower back; and
a plurality of cooling inserts configured for insertion into at least some of the plurality of pockets, wherein each cooling insert comprises a flexible thermal storage material.

11. The garment of claim 10, wherein each of the outer thigh pockets, buttocks pockets, stomach pocket and lower back pocket has a top-loading opening for insertion and removal of cooling inserts.

12. The garment of claim 11, wherein each of the inner thigh pockets has a side opening with a height substantially less than a height of the inner thigh pocket.

13. The garment of claim 10, wherein each inner thigh pocket, each outer thigh pocket, each buttocks pocket, and each corresponding cooling insert is approximately 6 inches (˜15 cm) wide and approximately 9 inches (˜23 cm) tall.

14. The garment of claim 10, wherein the stomach pocket and lower back pocket are configured to accommodate two cooling inserts approximately 6 inches (˜15 cm) tall and approximately 9 inches (˜23 cm) wide in a substantially side-by-side arrangement.

15. The garment of claim 1, wherein the plurality of cooling inserts are approximately 6 inches (˜15 cm) by approximately 9 inches (˜23 cm).

16. The garment of claim 13, wherein each inner thigh pocket and each outer thigh pocket are immediately adjacent one another along a substantial portion of their height, and each outer thigh pocket and each buttocks pocket are immediately adjacent one another.

17. The garment of claim 1, wherein each outer thigh pocket and each buttocks pocket are the same size and located immediately adjacent one another.

18. The garment of claim 1, wherein the flexible thermal storage material of each cooling insert comprises beads of phase change material that freeze at approximately 0° C. (32° F.) suspended in ethylene glycol.

19. The garment of claim 1, wherein at least one cooling insert is positioned in each of the plurality of pockets.

20. The garment of claim 14, wherein the stomach pocket and the lower back pocket are adjacent each other to extend substantially circumferentially around the waist of the wearer.

21. A method of applying cooling to fatty deposits of a wearer using the garment of claim 1 so as to reduce the fatty deposits, comprising:
freezing the cooling inserts in a flat position for at least two hours;
inserting the cooling inserts into the plurality of pockets adjacent to areas of the wearer having fatty deposits to be treated by cooling; and
wearing the garment for at least ½ hour.

22. A method of applying cooling to fatty deposits of a wearer using the garment of claim 10 so as to reduce the fatty deposits, comprising:
freezing the cooling inserts in a flat position for at least two hours;
inserting the cooling inserts into the plurality of pockets adjacent to areas of the wearer having fatty deposits to be treated by cooling; and
wearing the garment for at least ½ hour.

23. A method of treating fatty deposits of a user with cooling, comprising
positioning cooling means providing a first temperature on a garment at one or more locations corresponding to one or more fatty deposits to be treated;
positioning the cooling means adjacent the fatty deposits based upon the user wearing the garment; and
cooling the fatty deposits with the cooling means to a second temperature sufficient to stimulate brown fat to burn calories and compromise white fat cells by crystallization of cytoplasmic lipid deposits within the white fat cells.

24. The method of claim 23, wherein the first temperature is approximately 0° C.

25. The method of claim 24, wherein the cooling means comprises a flexible thermal storage material.

26. The method of claim 24 wherein the flexible thermal storage material comprises materials taken from the group consisting of inorganic matter and organic matter.
